(19) United States
(12) Patent Application Publication
(10) Pub. No.: US 2021/0007884 A1
(43) Pub. Date: Jan. 14, 2021

(54) METHOD AND APPARATUS FOR THERAPEUTIC HYPERTHERMIA DURING AN MRI SCAN

(71) Applicant: Aspect Imaging Ltd., Shoham (IL)

(72) Inventors: Uri RAPOPORT, Moshav Ben Shemen (IL); Noa STRAUSS, Peytah Tikva (IL); Shmuel AZULAY, Tel Aviv (IL)

(73) Assignee: Aspect Imaging Ltd., Shoham (IL)

(21) Appl. No.: 16/508,005

(22) Filed: Jul. 10, 2019

Related U.S. Application Data
(60) Provisional application No. 62/685,943.

(51) Int. Cl. A61F 7/00; A61B 5/055; G01R 33/50

(52) U.S. Cl. CPC A61F 7/0097; A61N 1/403; G01R 33/50; A61B 5/055

(57) ABSTRACT

A therapeutic hyperthermia device and method for using the same includes a cooling blanket dimensioned to at least partially cover a human, a conduit coupled to the cooling blanket, and cooling liquid. Each of the cooling blanket, conduit, and cooling liquid have a negligible effect on a magnetic resonance (MR) image taken by an MRI device. The cooling liquid may have a spin-lattice relaxation time (T1) and a spin-spin relaxation time (T2), where either or both of T1 and T2 are less than 2 milliseconds in duration. The cooling liquid may be one of water with manganite chloride, and water with sodium chloride.

### CROSS REFERENCE TO RELATED APPLICATIONS

This application claims priority to U.S. Provisional Patent Application No. 62/685,943 filed on Jul. 10, 2018, which is incorporated herein by reference in its entirety.

### FIELD

The present disclosure relates to the field of magnetic resonance (MR) imaging (MRI), and more particularly, to apparatus and methods for therapeutic hyperthermia during MR imaging.

### BACKGROUND

Hypoxic Ischemic Encephalopathy (HIE) is a type of brain damage that can occur when a human's brain doesn't receive sufficient levels of oxygen and/or blood. One treatment for HIE can include cooling the human's brain and/or body to reverse brain hypoxia. This cooling of the brain and/or body is commonly referred to as therapeutic hyperthermia. With respect to neonates, conventional therapeutic hyperthermia is typically continuous with no temperature rise (or substantially with no rise) of the neonate during the treatment process.

Current neonate therapeutic hypothermia methods include wrapping the neonate in a cooling blanket or laying the neonate on the blanket. These cooling blankets typically have small tubes with cooled water running there through.

It can be desirable to assess progression of the neonate's brain during therapeutic hyperthermia. But an MRI is not typically used for assessing progression of the neonate's brain during therapeutic hyperthermia due to, for example, logistical and safety concerns during patient transport from the neonatal intensive care unit (NICU) (e.g., extending tubing, moving a neonate to an MRI suite, coordinating standby staff in the MRI suite for medical emergencies during MRI, etc.), safety concerns due to incorporation of MR-unsafe materials in connection with the cooling blanket and peripheries (e.g., cooling units, connectors, etc.), and/or due to reduced MR image quality that can result due to conventional fluid (i.e., water in the cooling blanket.

Thus, there is a long felt need for a device capable of providing a therapeutic hyperthermia during MRI without negatively impacting MR image quality.

### SUMMARY

A therapeutic hyperthermia apparatus and method for using the same is disclosed. In one embodiment, the therapeutic hyperthermia apparatus includes a cooling blanket dimensioned to make contact with at least a portion of a human (e.g., a neonate), a conduit coupled to the cooling blanket, and a cooling liquid within the conduit. Each of the cooling blanket, conduit, and cooling liquid have a negligible effect on the MR image. In one embodiment, the cooling liquid has a spin-lattice relaxation time (T1) and a spin-spin relaxation time (T2). In one embodiment, either or both T1 and T2 are less than 2 milliseconds in duration.

In another embodiment, the therapeutic hyperthermia apparatus includes a cooling blanket dimensioned to make contact with at least a portion of a human (e.g., a neonate), a conduit coupled to the cooling blanket, cooling liquid, and a cooling unit. The cooling unit pumps the cooling liquid through the conduit and adjusts the temperature and/or flowrate of the cooling fluid based on a measured temperature of the human, the cooling blanket, and/or the cooling liquid. Each of the cooling blanket, conduit, and cooling liquid have a negligible effect on the MR image. In one embodiment, the cooling liquid has a spin-lattice relaxation time (T1) and a spin-spin relaxation time (T2). In one embodiment, either or both T1 and T2 are less than 2 milliseconds in duration.

In another embodiment, the therapeutic hyperthermia apparatus can be used with a superconducting magnet MRI or a permanent magnet MRI.

Additional aspects and advantages of the present disclosure are set forth in the detailed description which follows; possibly inferable from the detailed description; and/or learnable by practice of the present disclosure.

### BRIEF DESCRIPTION OF THE DRAWINGS

For a better understanding of embodiments of the disclosure and to show how the same can be carried into effect, reference will now be made, purely by way of example, to the accompanying drawings in which like numerals designate corresponding elements or sections throughout.

In the accompanying drawings:

FIGS. 1A, 1B and 1C are example schematic illustrations of therapeutic hyperthermia apparatus for a neonate;

FIG. 2 is a schematic illustration of one example of how the therapeutic hyperthermia apparatus (or components thereof) of FIG. 1B may be used with a superconducting magnet MRI device; and

FIG. 3 is a schematic illustration of one example of how the therapeutic hyperthermia apparatus (or components thereof) of FIG. 1B may be used with a permanent magnet MRI device.

It will be appreciated that, for simplicity and clarity of illustration, elements shown in the Figures have not necessarily been drawn to scale. For example, the dimensions of some of the elements can be exaggerated relative to other elements for clarity.

### DETAILED DESCRIPTION

In the following description, various aspects of the present disclosure are described. For purposes of explanation, specific configurations and details are set forth in order to provide a thorough understanding thereof. However, it will also be apparent to one skilled in the art that the disclosure can be practiced without the specific details presented herein. Furthermore, well known features can have been omitted or simplified in order not to obscure the present disclosure. With specific reference to the drawings, the particulars shown are by way of example and for purposes of illustrative discussion of the present disclosure only and are presented in the cause of providing what is believed to be the most useful and readily understood description of the principles and conceptual aspects of the disclosure. In this regard, no attempt is made to show structural details in more detail than is necessary for a fundamental understanding of the disclosure, the description taken with the drawings making apparent to those skilled in the art how the several forms of the disclosure can be embodied in practice.

Before at least one embodiment of the disclosure is explained in detail, it is to be understood that the disclosure is not limited in its application to the details of construction and the arrangement of the components set forth in the following description or illustrated in the drawings. The disclosure is applicable to other embodiments that can be practiced or carried out in various ways as well as to combinations of the disclosed embodiments. Also, it is to be understood that the phraseology and terminology employed herein is for the purpose of description and should not be regarded as limiting.

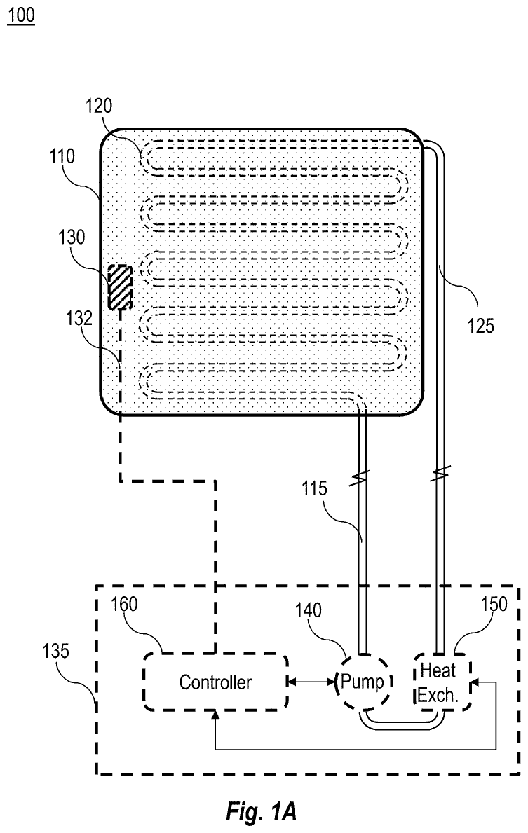

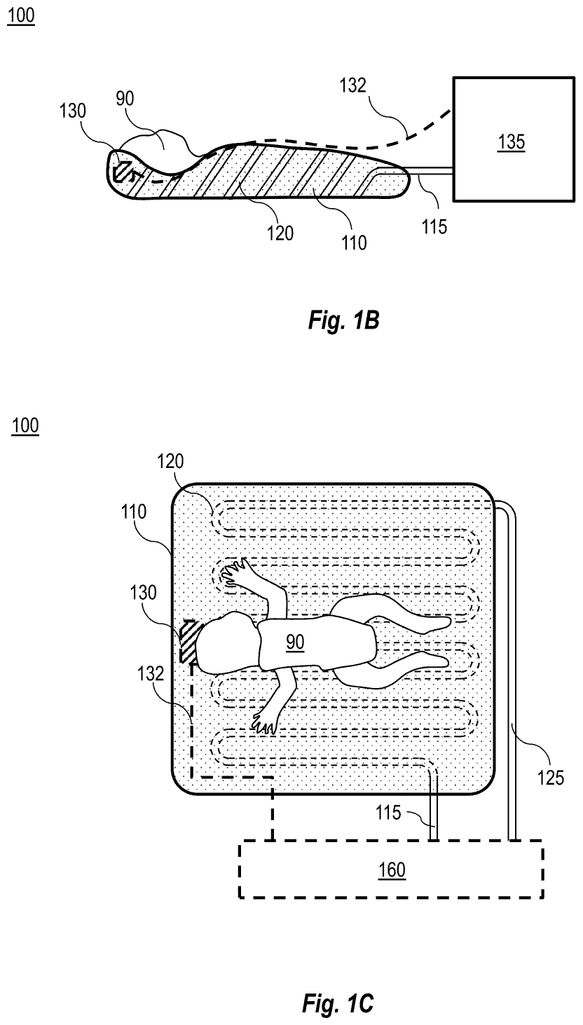

Reference is now made to FIGS. 1A, 1B and 1C, which are exemplary schematic illustrations of hyperthermia apparatus 100 for a neonate 90 capable of being used during MR imaging. Apparatus 100 includes a cooling blanket 110, a conduit 120, and cooling fluid (not separately depicted). Each of the cooling blanket 110, conduit 120, and cooling fluid can have a negligible effect on an MR image and thus can enable the MR imaging of the neonate during the therapeutic hyperthermia thereof (e.g., as described below).

Cooling blanket 110 can be dimensioned to make contact with at least a portion of neonate 90. For example, in FIG. 1B, the cooling blanket 110 can envelope at least a portion of neonate 90 and in FIG. 1C, neonate 90 can be placed on blanket 90. Cooling blanket 110 and conduit 120 can be made of a plastic polymer such as polyvinyl chloride (PVC), polyethylene (PE) and polypropylene (PP).

In one embodiment, cooling blanket 110 can have two external surfaces (i.e., a top and a bottom). The conduit 120 (e.g., a tube network) may be coupled to at least a portion of one or both of the two external surfaces. In one embodiment, the location of the coupling of the conduit 120 to the at least a portion of one or both of the two external surfaces is changeable.

In another embodiment, cooling blanket 110 can have two external surfaces (i.e., a top and a bottom) and two internal surfaces (i.e., the back-side to a top external surface and a back-side to a bottom external surface). The conduit 120 (e.g., a tube network) may be coupled to at least a portion of one or more of the two internal surfaces. In one embodiment, the location of the coupling of the conduit 120 to the portion of one of the two internal surfaces is changeable.

In another embodiment, the conduit 120 is integrally formed within the cooling blanket 110 (e.g., as part of the manufacturing process of the cooling blanket 110). For example, if the cooling blanket 110 is comprised of two plastic polymer sheets adhered to one another at a plurality of locations, then at least a portion of the non-adhered locations can form one or more channels that make up a conduit 120.

The cooling liquid can be a liquid that has a negligible effect on an MRI imaging by an MRI device. For example, the cooling liquid can have a negligible effect on a dynamic range of an intensity of an MR image obtained by an MRI device and/or does not distort (or substantially not distort) the image thereof.

In various embodiments, a spin-lattice relaxation time (T1), a spin-spin relaxation time (T2), or both relaxation times T1 and T2 of the cooling liquid are less than 2 milliseconds in duration (e.g., such that the cooling liquid is not visible to an MR imaging by an MRI device). For example, the cooling liquid can be water (e.g., distilled water) with manganite-chloride. In another example, the cooling liquid can be water (e.g., is distilled water) and sodium chloride.

In one embodiment, one or more temperature sensors 130 can be coupled to cooling blanket 110. Temperature sensor(s) 130 can measure a temperature of at least a portion of neonate 90, the cooling blanket 110, and the cooling liquid. Temperature sensor(s) 130 can have a negligible effect on an MRI imaging by an MRI device. In some embodiments, temperature sensor(s) 130 is fiber optic temperature sensor.

In some embodiments, therapeutic hypothermia apparatus 100 includes cooling unit 135 coupled to the cooling blanket 110. In one embodiment, cooling unit 135 includes pump 140 and heat exchanger 150. Pump 140 and/or heat exchanger 150 can be in fluid communication with conduit 120 (e.g., via a supply conduit 115 and a return conduit 125). Pump 140 can circulate cooling liquid through supply conduit 115 and conduit 120, back to the cooling unit 135 via return conduit 120 at a predetermined or configurable flowrate value. Heat exchanger 150 can cool and/or heat the cooling liquid to a predetermined or configurable temperature to cause the neonate 90, cooling blanket 110 and/or conduit to exhibit a desired temperature.

In one embodiment, cooling unit 135 includes controller 160. Controller 160 controls pump 140 and heat exchanger 150. For example, controller 160 can control the pump 140 and heat exchanger 150 to operate at predetermined or configurable values to maintain or adjust flowrate and temperature of the cooling liquid. In one embodiment, the controller 160 is in communication with temperature sensor(s) 130 attached to cooling blanket 110. For example, temperature sensor(s) 130 can be in communication with controller 160 via cable 132 (e.g., a fiber optic cable). In another embodiment temperature sensor(s) 130 is in communication with controller 160 using a wireless technology, as is understood by one of ordinary skill in the art. Controller 160 controls pump 140 and heat exchanger 150 based on the temperature(s) measured by the temperature sensor(s) 130. In another embodiment, controller 160 controls pump 140 and heat exchanger 150 based on a property (e.g., flowrate and temperature) of the cooling liquid as measured by the cooling unit (e.g., at the return conduit 125).

In one embodiment, cooling unit 135 includes a cooling fluid reservoir (not depicted). Cooling fluid can be introduced into therapeutic hyperthermia device 100 (or replaced) by way of the reservoir. In operation, the cooling unit 135 in such embodiment might pump cooling liquid through supply conduit, conduit, and back to the cooling unit 135 via return conduit 125. As cooling liquid returns to the cooling unit 135, it can be stored in the cooling fluid reservoir where heat exchanger 150 may adjust the temperature of such stored cooling fluid before it is returned to the cooling blanket 110 using pump 140.

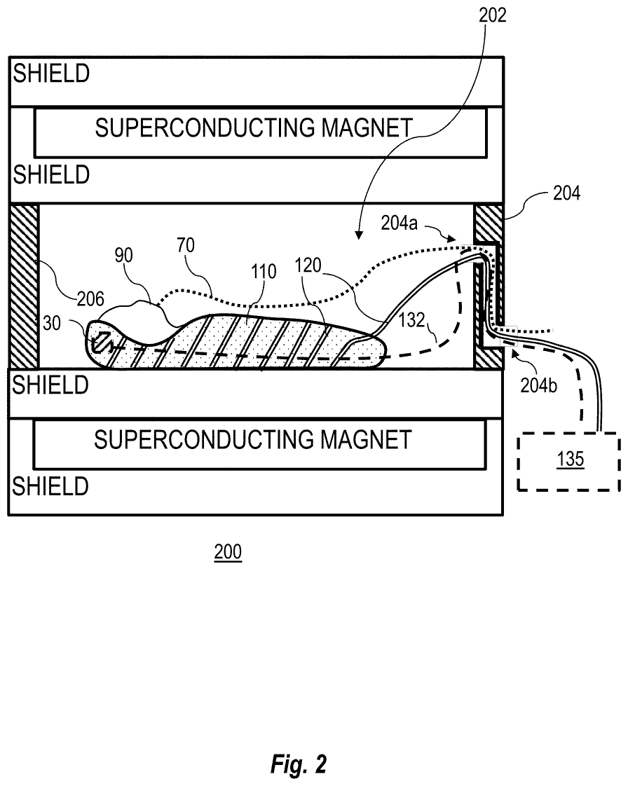

Reference is now made to FIG. 2, which is a schematic illustration of one example of how the therapeutic hyperthermia apparatus 100 (or components thereof) of FIG. 1B may be used with a superconducting magnet MRI device 200. In this example, neonate 90 is enveloped by cooling blanket 110 of therapeutic hyperthermia apparatus 100, and inserted into a bore 202 of MRI device 200. Therapeutic hyperthermia apparatus 100 may be part of a system that includes a first magnetic and radiofrequency (RF) shield door 204 and a second magnetic and RF shield door 206. Doors 204 and 206 can be made of a material that substantially shields magnetic (e.g., B0) and RF (e.g., B1) fields. Doors 204 and 206 can be retrofit onto existing superconductor magnet MRI devices 200, as depicted. In other embodiments, doors 204 and/or door 206 is/are replaced with magnetic and RF shielding sleeve (e.g., material comprising a faraday cage) to substantially shield magnetic and RF fields.

In some embodiments, door 204 includes a conduit having a length to width ratio (e.g., 5:1). In one embodiment, the conduit starts at a first aperture 204a and extends to a second aperture 204a. The conduit can enable introduction of medical tubing/cables (e.g., such as supply conduit 115, return conduit 125, cable 132 and/or other medical connectors/tubes 70 connected to neonate 90) into bore 202 of superconducting magnet MRI device 200 while eliminating (or substantially eliminating) RF fields from exiting/entering bore 200 of superconducting magnet MRI device 200. In some embodiments, the conduit is horizontal within the incubator and exits the incubator and the MRI device via an aperture in each.

According to various embodiments, T1W, T2W, DWI sequences as are known in the art, and/or SE, FSE, EPI GRE susceptibility weighted imaging can be used by superconducting magnet MRI device 200 when imaging neonate 90 during therapeutic hyperthermia provided by apparatus 100. In this manner therapeutic hyperthermia can be provided to neonate 90 by apparatus 100 during an MRI scan by superconducting magnet MRI device 200 without causing a leakage of RF radiation from/into superconducting magnet MRI device 200.

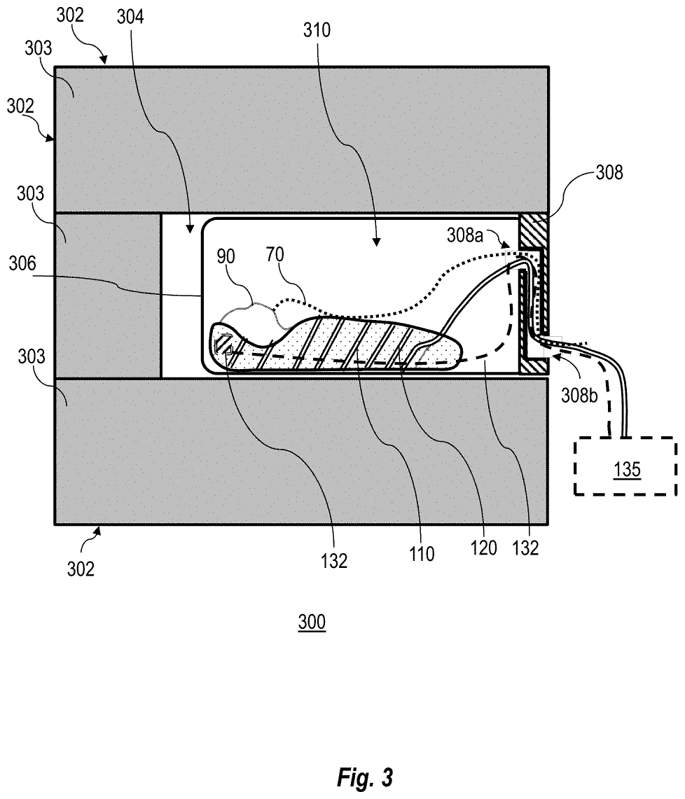

Reference is now made to FIG. 3, which is a schematic illustration of one example of how the therapeutic hyperthermia apparatus 100 (or components thereof) of FIG. 1B may be used with a permanent magnet MRI device 300. In this example, permanent magnet MRI device 300 may include permanent magnet housing 302, permanent magnet(s) 303, and bore 304. Permanent magnet housing 302 may be made of a material that shields an environment exterior to permanent magnet MRI device 300 from the magnetic fields (e.g., magnetic fringe fields) generated by magnets, such as permanent magnets 303, within permanent magnet MRI device 210 and RF energy generated by one or more RF coils (not depicted) within or inserted into permanent magnet MRI device 200. Housing 302 of permanent magnet MRI device 300 can also prevent magnetic fields and RF energy exterior to permanent magnet MRI device 300 from entering permanent magnet MRI device 300, and thus causing interference in the imaging results. Permanent magnet MRI device 300 can be a permanent magnet-based MRI as described in U.S. Pat. Nos. 7,400,147 and/or 7,315,168, both of which are incorporate herein by reference in their entireties.

In one embodiment, capsule incubator 306 includes an interior 310 dimensioned to receive neonate 90. Capsule incubator 306 can include an RF shield 308 at, for example, one of ends of capsule incubator 306. RF shield 308 can include at least two apertures 308a, 308b and a conduit (not numbered) extending between apertures 308a, 308b. RF shield 308 can eliminate (or substantially eliminate) RF waves from entering/exiting capsule incubator 220 despite apertures 308a, 308b. RF shield 306 can mate with bore 304. When capsule incubator 306 is positioned within bore 304, the walls of bore 304 enclose conduit to form a conduit that is completely (or substantially completely) closed.

Therapeutic hyperthermia apparatus 100 can be employed in permanent magnet MRI device 200 in a manner similar to that described above with respect to superconducting magnet MRI device 200 of FIG. 2. In one embodiment, therapeutic hyperthermia apparatus 100 may be part of a system that includes capsule incubator 306. Neonate 90 can be at least partly enveloped by cooling blanket 110 (or placed thereon) and inserted into interior 310 of capsule incubator 306. Capsule incubator 306 can be inserted into bore 304 of permanent magnet MRI device 300 while conduit between apertures 308a and 308b can permit medical tubing and cables (e.g., supply conduit 115, return conduit 125, cable 132 and/or other medical connectors/tubes 70 connected to neonate 90) to pass from the interior 132 of capsule incubator 306 to the space external to permanent magnet MRI device 300. According to various embodiments, regular T1W, T2W, DWI sequences and/or SE, FSE, EPI GRE susceptibility weighted imaging can be used by permanent magnet MRI device 300 when imaging neonate 90 during therapeutic hyperthermia provided by apparatus 100. In this manner, therapeutic hyperthermia can be provided to neonate 90 by apparatus 100 during MR imaging by permanent magnet MRI device 300 without causing a leakage of RF radiation from/into permanent magnet MRI device 300.

Advantageously, the disclosed apparatus and methods of using the same as described above can enable therapeutic hyperthermia of neonate 90 during an MR imaging by an MRI device without a negative image on MR image quality. In this manner, the disclosed apparatus can enable monitoring a brain damage (e.g., Hypoxic Ischemic Encephalopathy) of neonate 90 by an MRI device without a need to terminate the therapeutic hyperthermia.

Providing a cooling blanket can be done by a manufacturer of the cooling blanket, a hospital, doctor or any provider of the cooling blanket.

In the above description, an embodiment is an example or implementation of the description. The various appearances of “one embodiment”, “an embodiment”, “certain embodiments” or “some embodiments” do not necessarily all refer to the same embodiments. Although various features of the description can be described in the context of a single embodiment, the features can also be provided separately or in any suitable combination. Conversely, although the description can be described herein in the context of separate embodiments for clarity, the description can also be implemented in a single embodiment. Certain embodiments of the description can include features from different embodiments disclosed above, and certain embodiments can incorporate elements from other embodiments disclosed above. The disclosure of elements of the description in the context of a specific embodiment is not to be taken as limiting their use in the specific embodiment alone. Furthermore, it is to be understood that the description can be carried out or practiced in various ways and that the description can be implemented in certain embodiments other than the ones outlined in the description above.

The description is not limited to those diagrams or to the corresponding descriptions. For example, flow need not proceed strictly in the order as illustrated or described herein. Meanings of technical and scientific terms used herein are to be commonly understood as by one of ordinary skill in the art to which the description belongs, unless otherwise defined. While the description has been described with respect to a limited number of embodiments, these should not be construed as limitations on the scope of the description, but rather as exemplifications of some of the preferred embodiments. Other possible variations, modifications, and applications are also within the scope of the description. Accordingly, the scope of the description should not be limited by what has thus far been described, but by the appended claims and their legal equivalents.

1. An apparatus for therapeutic hyperthermia of a human during a magnetic resonance (MR) imaging (MRI) by an MRI device, the apparatus comprising:
a cooling blanket dimensioned to make contact with at least a portion of the human;
a conduit coupled to the cooling blanket; and
a cooling liquid within the conduit, wherein:
each of the cooling blanket, conduit, and cooling liquid have a negligible effect on an MR image.

2. The apparatus of claim 1, wherein the cooling blanket comprises one of:
two external surfaces and the conduit is coupled to at least one external surface; and
two internal surfaces and the conduit is coupled to at least one internal surface.

3. The apparatus of claim 1, wherein the conduit is integrally formed within the cooling blanket.

4. The apparatus of claim 1, wherein the cooling liquid has a spin-lattice relaxation time (T1) and a spin-spin relaxation time (T2), and where at least one of T1 and T2 are less than 2 milliseconds in duration.

5. The apparatus of claim 1, wherein the cooling liquid is one of: water with manganite chloride, and water with sodium chloride.

6. The apparatus of claim 1, further comprising a temperature sensor coupled to the cooling blanket, the temperature sensor being operative to measure a temperature of at least one of: the human, the cooling blanket, and the cooling liquid, wherein the temperature sensor has a negligible effect on the MR image.

7. The apparatus of claim 1, wherein the temperature sensor is a fiber optic sensor.

8. The apparatus of claim 1, further comprising a cooling unit, wherein the cooling unit is operative to:
pump the cooling liquid through the conduit; and
adjust at least one of:
a temperature of the cooling fluid based on a measured temperature of at least one of: the human, the cooling blanket, and the cooling liquid; and
a flowrate of the cooling fluid based on the measured temperature.

9. A method for therapeutic hyperthermia of a human during a magnetic resonance (MR) imaging (MRI) by an MRI device, the method comprising:
providing a cooling blanket with a conduit, wherein the cooling blanket is dimensioned to make contact with at least a portion of the human; and
pumping a cooling liquid through the conduit, wherein each of the cooling blanket, conduit, and cooling liquid have a negligible effect on an MR image.

10. The method of claim 9, wherein the cooling liquid has a spin-lattice relaxation time (T1) and a spin-spin relaxation time (T2), and where at least one of T1 and T2 are less than 2 milliseconds in duration.

11. The method of claim 9, wherein the cooling liquid is one of: water with manganite chloride, and water with sodium chloride.

12. The method of claim 9, further comprising measuring, using a temperature sensor coupled to the cooling blanket, the temperature sensor a temperature of at least one of: the human, the cooling blanket, and the cooling liquid, wherein the temperature sensor has a negligible effect on the MR image.

13. The method of claim 9, further comprising adjusting at least one of:
a temperature of the cooling fluid based on a measured temperature of at least one of: the human, the cooling blanket, and the cooling liquid; and
a flowrate of the cooling fluid based on the measured temperature.

14. An apparatus for therapeutic hyperthermia of a human during a magnetic resonance (MR) imaging (MRI) by an MRI device, the apparatus comprising:
a cooling blanket dimensioned to make contact with at least a portion of the human;
a conduit coupled to the cooling blanket; and
a cooling liquid,
a cooling unit operative to:
pump the cooling liquid through the conduit; and
adjust at least one of:
a temperature of the cooling fluid based on a measured temperature of at least one of: the human, the cooling blanket, and the cooling liquid; and
a flowrate of the cooling fluid based on the measured temperature;
wherein each of the cooling blanket, conduit, and cooling liquid have a negligible effect on an MR image.

15. The apparatus of claim 14, wherein the cooling liquid has a spin-lattice relaxation time (T1) and a spin-spin relaxation time (T2), and where at least one of T1 and T2 are less than 2 milliseconds in duration.

16. The apparatus of claim 14, wherein the cooling liquid is one of: water with manganite chloride, and water with sodium chloride.

17. The apparatus of claim 14, further comprising a temperature sensor coupled to the cooling blanket, the temperature sensor being operative to provide the measured temperature, wherein the temperature sensor has a negligible effect on the MR image.

18. A method for therapeutic hyperthermia of a human during a magnetic resonance (MR) imaging (MRI) by an MRI device, the method comprising:
providing a cooling blanket with a conduit, wherein the cooling blanket is dimensioned to make contact with at least a portion of the human;
pumping a cooling liquid through the conduit; and
adjust at least one of:
a temperature of the cooling fluid based on a measured temperature of at least one of: the human, the cooling blanket, and the cooling liquid; and
a flowrate of the cooling fluid based on the measured temperature,
wherein each of the cooling blanket, conduit, and cooling liquid have a negligible effect on an MR image.

19. The method of claim 18, wherein the cooling liquid has a spin-lattice relaxation time (T1) and a spin-spin relaxation time (T2), and where at least one of T1 and T2 are less than 2 milliseconds in duration.

20. The method of claim 18, wherein the cooling liquid is one of: water with manganite chloride, and water with sodium chloride.
